(19) United States
(12) Patent Application Publication
(10) Pub. No.: US 2020/0323584 A1
(43) Pub. Date: Oct. 15, 2020

(54) ABLATION CATHETER TIP WITH FLEXIBLE ELECTRONIC CIRCUITRY

(71) Applicant: St. Jude Medical International Holding S.à r.l., Luxembourg (LU)

(72) Inventors: Jacob J. Daly, Ham Lake, MN (US); Brett A. Hillukka, Hanover, MN (US)

(21) Appl. No.: 16/844,554

(22) Filed: Apr. 9, 2020

Related U.S. Application Data
(60) Provisional application No. 62/832,246, filed on Apr. 10, 2019.

(51) Int. Cl. A61B 18/14

(52) U.S. Cl. CPC A61B 18/1492; A61B 2018/00577

(57) ABSTRACT

Aspects of the present disclosure are directed to, for example, a high-thermal-sensitivity ablation catheter tip with force measurement capability. More specifically, various aspects of the present disclosure are directed to improving the deformation consistency of the ablation catheter tip in response to various forces, and thereby improving force measurement accuracy of an ablation catheter system.

### CROSS-REFERENCE TO RELATED APPLICATIONS

This application claims the benefit of U.S. provisional application No. 62/832,246, filed 10 Apr. 2019, which is hereby incorporated by reference as though fully set forth herein.

This application incorporates by reference as though fully set forth herein: U.S. application Ser. No. 15/088,036, filed 31 Mar. 2016, now pending, which claims the benefit of U.S. provisional application No. 62/141,066, filed 31 Mar. 2015; U.S. application Ser. No. 15/088,052, filed 31 Mar. 2016, now pending, which claims the benefit of U.S. provisional application No. 62/198,114, filed 28 Jul. 2015; U.S. application Ser. No. 15/723,701, filed 3 Oct. 2017, now pending, which claims the benefit of U.S. provisional application No. 62/404,038, filed 4 Oct. 2016; U.S. application Ser. No. 15/724,157, filed 3 Oct. 2017, now pending, which claims the benefit of U.S. provisional application No. 62/404,060, filed 4 Oct. 2016; international application no. PCT/US2017/049264, filed 30 Aug. 2017, now pending, which claims the benefit of U.S. provisional application No. 62/404,013, filed 4 Oct. 2016; U.S. provisional application No. 62/642,178, filed 13 Mar. 2018; U.S. provisional application No. 62/824,840, filed 27 Mar. 2019; U.S. provisional application No. 62/824,844, filed 27 Mar. 2019; and U.S. provisional application No. 62/824,846, filed 27 Mar. 2019.

### BACKGROUND OF THE DISCLOSURE

### a. Field

The instant disclosure relates to various types of medical catheters, in particular catheters for diagnostics within, and/or treatment of, a patient's cardiovascular system. In one embodiment, the instant disclosure relates to an ablation catheter for treating cardiac arrhythmias within a cardiac muscle. Various aspects of the instant disclosure relate to force sensing systems capable of determining a force applied at a distal tip of the ablation catheter.

The present disclosure further relates to low thermal mass ablation catheter tips (also known as high-thermal-sensitivity catheter tips) and to systems for controlling the delivery of RF energy to such catheters during ablation procedures.

### b. Background

Exploration and treatment of various organs or vessels has been made possible using catheter-based diagnostic and treatment systems. These catheters may be introduced through a vessel leading to the cavity of the organ to be explored, and/or treated. Alternatively, the catheter may be introduced directly through an incision made in the wall of the organ. In this manner, the patient avoids the trauma and extended recuperation times typically associated with open surgical procedures.

The human heart routinely experiences electrical currents traversing its many layers of tissue. Just prior to each heart contraction, the heart depolarizes and repolarizes as electrical currents spread across the heart. In healthy hearts, the heart will experience an orderly progression of depolarization waves. In unhealthy hearts, such as those experiencing atrial arrhythmia, including for example, ectopic atrial tachycardia, atrial fibrillation, and atrial flutter, the progression of the depolarization wave becomes chaotic.

Catheters are used in a variety of diagnostic and/or therapeutic medical procedures to diagnose and correct conditions such as atrial arrhythmia. Typically, in such a procedure, a catheter is manipulated through a patient's vasculature to the patient's heart carrying one or more end effectors which may be used for mapping, ablation, diagnosis, or other treatment. Where an ablation therapy is desired to alleviate symptoms including atrial arrhythmia, an ablation catheter imparts ablative energy to cardiac tissue to create a lesion in the cardiac tissue. The lesioned tissue is less capable of conducting electrical signals, thereby disrupting undesirable electrical pathways and limiting or preventing stray electrical signals that lead to arrhythmias. The ablation catheter may utilize ablative energy including, for example, radio frequency (RF), cryoablation, laser, chemical, and high-intensity focused ultrasound. Ablation therapies often require precise positioning of the ablation catheter, as well as precise pressure exertion for optimal ablative-energy transfer into the targeted myocardial tissue. Excess pressure between the ablation catheter tip and the targeted myocardial tissue may result in excessive ablation which may permanently damage the cardiac muscle and/or surrounding nerves. When the contact pressure between the ablation catheter tip and the targeted myocardial tissue is below a target pressure, the efficacy of the ablation therapy may be reduced.

Ablation therapies are often delivered by making a number of individual ablations in a controlled fashion in order to form a lesion line. To improve conformity of the individual ablations along the lesion line, it is desirable to precisely control the position at which the individual ablations are conducted, the ablation period, and the contact pressure between the ablation catheter tip and the targeted tissue. All of these factors affect the conformity of the resulting lesion line.

The foregoing discussion is intended only to illustrate the present field and should not be taken as a disavowal of claim scope.

### BRIEF SUMMARY OF THE DISCLOSURE

It is desirable to control the delivery of RF energy to a catheter to enable the creation of lesions in tissue, by keeping the generator power setting sufficiently high to form adequate lesions, while mitigating against overheating of tissue. Accordingly, aspects of the present disclosure are directed toward an ablation catheter tip including high thermal sensitivity materials which facilitate near real-time temperature sensing at the ablation catheter tip. Further aspects of the present disclosure are directed to improved ablation catheter force measurements in response to tissue contact on the ablation catheter tip.

One embodiment of the present disclosure is directed to a high thermal-sensitivity ablation catheter tip. The high-thermal-sensitivity ablation catheter tip includes a conductive shell, a structural member, a manifold, and a flexible electronic circuit. The conductive shell includes a dispersion chamber for irrigant distribution. The structural member is coupled to a proximal end of the conductive shell, and deflects in response to a force exerted on the conductive shell. The manifold includes an irrigation lumen extending through a longitudinal axis of the manifold, and the irrigation lumen delivers irrigant into the dispersion chamber. The flexible electronic circuit extends through the irrigation lumen of the manifold. In more specific embodiments, the flexible electronic circuit includes one or more bends positioned on a portion of the flexible circuit within the irrigant lumen. The one or more bends deflect in response to an axial force exerted on the conductive shell while minimally absorbing the axial force.

Some embodiments of the present disclosure are directed to a method of assembling an ablation catheter tip. One example of such a method includes the following steps: providing a manifold with an irrigant lumen extending there through; providing a flexible electronic circuit including one or more thermocouples; and directing a distal portion of the flexible circuit through the irrigant lumen. In more specific embodiments, the method further includes forming a bend in the flexible electronic circuit, and positioning the bend within the irrigant lumen of the manifold.

The foregoing and other aspects, features, details, utilities, and advantages of the present disclosure will be apparent from reading the following description and claims, and from reviewing the accompanying drawings.

### DESCRIPTION OF THE DRAWINGS

Various example embodiments may be more completely understood in consideration of the following detailed description in connection with the accompanying drawings.

FIG. 1 is a diagrammatic overview of an ablation catheter system including a force sensing subsystem, consistent with various embodiments of the present disclosure;

FIG. 2 is a top view of a flexible electronic circuit (also referred to as a flexible circuit), consistent with various aspects of the present disclosure.

FIG. 3 depicts an isometric front view of a partial catheter tip assembly including a tip insert with a flexible electronic circuit wrapped circumferentially around the tip insert, consistent with various aspects of the present disclosure.

FIG. 4 depicts an isometric front view, including a partial cut-out, of the partial catheter tip assembly of FIG. 3 with a conductive shell encompassing at least a portion of the tip insert and the flexible electronic circuit, consistent with various aspects of the present disclosure.

FIG. 5A is an isometric front view of an ablation catheter tip assembly, consistent with various aspects of the present disclosure.

FIG. 5B is a cross-sectional, isometric front view of the ablation catheter tip assembly of FIG. 5A, consistent with various aspects of the present disclosure.

FIG. 5C is a cross-sectional front view of the ablation catheter tip assembly of FIG. 5A, with the ablation catheter tip assembly coupled to a distal end of a catheter shaft, consistent with various aspects of the present disclosure.

While various embodiments discussed herein are amenable to modifications and alternative forms, aspects thereof have been shown by way of example in the drawings and will be described in detail. It should be understood, however, that the intention is not to limit the invention to the particular embodiments described. On the contrary, the intention is to cover all modifications, equivalents, and alternatives falling within the scope of the disclosure including aspects defined in the claims. In addition, the term “example” as used throughout this application is only by way of illustration, and not limitation.

### DETAILED DESCRIPTION OF EMBODIMENTS

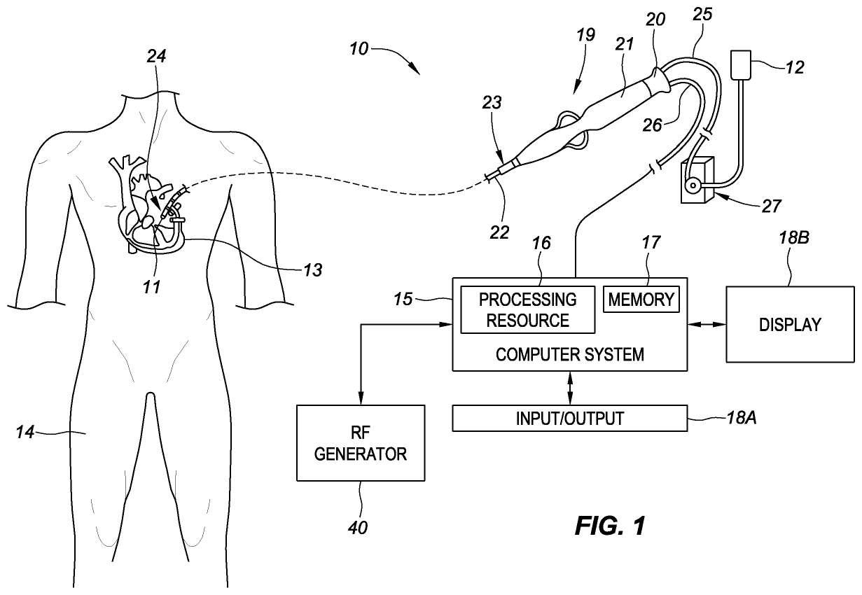

Referring now to the drawings wherein like reference numerals are used to identify identical components in the various views, FIG. 1 generally illustrates an ablation catheter system 10 having an elongated medical device 19 that includes a sensor assembly 11 (e.g., fiber optic based distance measurement sensor) configured to be used in the body for medical procedures. The elongated medical device 19 may be used for diagnosis, visualization, and/or treatment of tissue 13 (such as cardiac or other tissue) in the body. For example, the medical device 19 may be used for ablation therapy of tissue 13 or mapping of a patient's body 14. FIG. 1 further illustrates various sub-systems included in the ablation catheter system 10. The system 10 may include a main computer system 15 (including an electronic control unit 16 and data storage 17, e.g., memory). The computer system 15 may further include conventional interface components, such as various user input/output mechanisms 18A and a display 18B, among other components. Information provided by the sensor assembly 11 may be processed by the computer system 15 and may provide data to the clinician via the input/output mechanisms 18A and/or the display 18B, or in other ways as described herein. Specifically, the display 18B may visually communicate a force exerted on the elongated medical device 19—where the force exerted on the elongated medical device 19 is detected in the form of a deformation of at least a portion of the elongated medical device by the sensor assembly 11, and the measured deformation is processed by the computer system 15 to determine the force exerted.

In the illustrative embodiment of FIG. 1, the elongated medical device 19 may include a cable connector or interface 20, a handle 21, a tubular body or shaft 22 having a proximal end 23 and a distal end 24. The elongated medical device 19 may also include other conventional components not illustrated herein, such as a temperature sensor, additional electrodes, and corresponding conductors or leads. The connector 20 may provide mechanical, fluid and/or electrical connections for cables 25, 26 extending from a fluid reservoir 12 and a pump 27 and the computer system 15, respectively. The connector 20 may comprise conventional components known in the art and, as shown, may be disposed at the proximal end of the elongated medical device 19.

The handle 21 provides a portion for a user to grasp or hold the elongated medical device 19 and may further provide a mechanism for steering or guiding the shaft 22 within the patient's body 14. For example, the handle 21 may include a mechanism configured to change the tension on a pull-wire extending through the elongated medical device 19 to the distal end 24 of the shaft 22 or some other mechanism to steer the shaft 22. The handle 21 may be conventional in the art, and it will be understood that the configuration of the handle 21 may vary. In an embodiment, the handle 21 may be configured to provide visual, auditory, tactile and/or other feedback to a user based on information received from the sensor assembly 11. For example, if contact to tissue 13 is made by distal end 24, the sensor assembly 11 may transmit data to the computer system 15 indicative of contact. In response to the computer system 15 determining that the data received from the sensor assembly 11 is indicative of contact between the distal end 24 and a patient's body 14, the computer system 15 may operate a light-emitting-diode on the handle 21, a tone generator, a vibrating mechanical transducer, and/or other indicator(s), the outputs of which could vary in proportion to the calculated contact force.

The computer system 15 may utilize software, hardware, firmware, and/or logic to perform a number of functions described herein. The computer system 15 may be a combination of hardware and instructions to share information. The hardware, for example may include processing resource 16 and/or a memory 17 (e.g., non-transitory computer-readable medium (CRM) database, etc.). A processing resource 16, as used herein, may include a number of processors capable of executing instructions stored by the memory resource 17. Processing resource 16 may be integrated in a single device or distributed across multiple devices. The instructions (e.g., computer-readable instructions (CRI)) may include instructions stored on the memory 17 and executable by the processing resource 16 for force detection.

The memory resource 17 is communicatively coupled with the processing resource 16. A memory 17, as used herein, may include a number of memory components capable of storing instructions that are executed by processing resource 16. Such a memory 17 may be a non-transitory computer readable storage medium, for example. The memory 17 may be integrated in a single device or distributed across multiple devices. Further, the memory 17 may be fully or partially integrated in the same device as the processing resource 16 or it may be separate but accessible to that device and the processing resource 16. Thus, it is noted that the computer system 15 may be implemented on a user device and/or a collection of user devices, on a mobile device and/or a collection of mobile devices, and/or on a combination of the user devices and the mobile devices.

The memory 17 may be communicatively coupled with the processing resource 16 via a communication link (e.g., path). The communication link may be local or remote to a computing device associated with the processing resource 16. Examples of a local communication link may include an electronic bus internal to a computing device where the memory 17 is one of a volatile, non-volatile, fixed, and/or removable storage medium in communication with the processing resource 16 via the electronic bus.

In various embodiments of the present disclosure, the computer system 15 may receive optical signals from a sensor assembly 11 via one or more optical fibers extending a length of the catheter shaft 22. A processing resource 16 of the computer system 15 may execute an algorithm stored in memory 17 to compute a force exerted on distal end 24, based on the received optical signals.

U.S. Pat. No. 8,567,265 discloses various optical force sensors for use in medical catheter applications, such optical force sensors are hereby incorporated by reference as though fully disclosed herein.

FIG. 1 further depicts an RF generator 40 operatively connected to the computer system 15, which is operatively connected to the elongated medical device 19. In this figure, a number of possible wired and/or wireless communication pathways are shown. For example, the computer system 15 may receive temperature feedback readings from at least one temperature sensor mounted on or near the distal end 24 of the catheter shaft 22. In various embodiments disclosed herein, the catheter may include multiple thermal sensors (for example, thermocouples or thermistors), as described further below. The temperature feedback readings may be the highest reading from among all of the individual temperature sensor readings, or it may be, for example, an average of all of the individual readings from all of the temperature sensors. The computer system 15 may then communicate to the RF generator 40 the highest temperature measured by any of the plurality of temperature sensors mounted within the sensor assembly 11. This could be used to trigger a temperature-based shutdown feature in the RF generator for patient safety. In other words, the temperature reading or readings from the catheter may be sent to the computer system 15, which may then feed the highest temperature reading to the RF generator 40 so that the generator can engage its safety features and shut down (or titrate power) if the temperature reading exceeds a (safety) threshold.

In an alternative operation of the system 10 of FIG. 1, the computer system 15, in response to elevated temperature feedback from the thermal sensors, may operate the RF generator 40 in a pulsed manner. By pulsing the RF signal, the power can remain at a desired power level (e.g., 50 or 60 Watts) rather than being reduced to an ineffective level when excessive temperature is sensed by the catheter tip. In particular, rather than reducing the power to control temperature, the power may be delivered in a pulsed manner; by pulsing the RF signals, and controlling the length of pulse and gaps between pulses, tip temperature may be controlled. Similar to that described above, instead of pulsing the power, the power may also be titrated in such a manner.

In the embodiment depicted in FIG. 1, the RF generator 40 may include pulse control hardware, software, and/or firmware built into the generator itself.

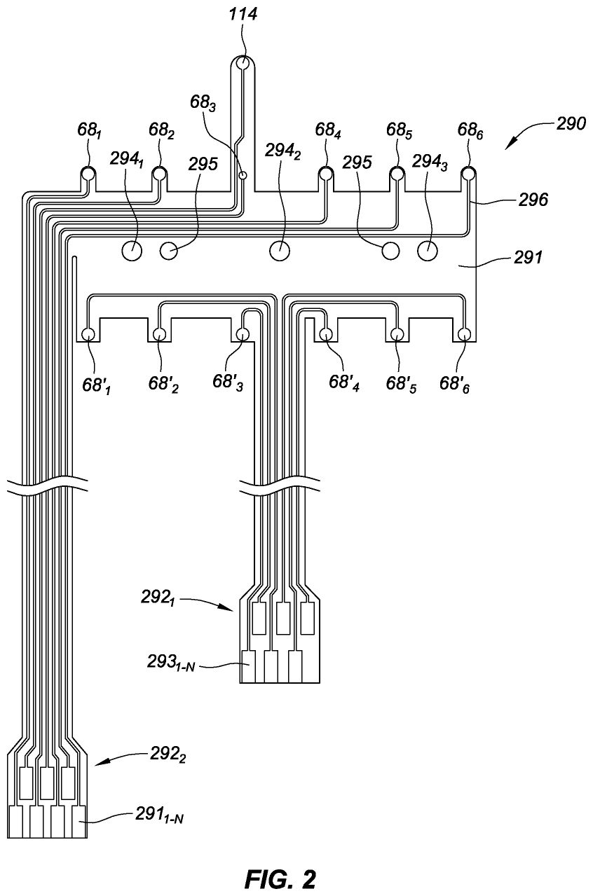

FIG. 2 is a top view of a flexible circuit 290, consistent with various aspects of the present disclosure. In various embodiments, the flexible circuit 290 may be installed on a tip insert of a catheter tip assembly instead of utilizing individually wired temperature sensors and electrophysiology electrodes. By consolidating the various wire leads into one or more flexible circuits, or even one or more flexible circuits plus a few wire leads, the cost, complexity, and manufacturing assembly time associated with such ablation tip assemblies may be greatly reduced.

Flexible circuit 290 may include one or more connectors 292 located at the distal end of a strand of the flexible circuit to facilitate manufacturability within a catheter tip sub-assembly. For example, where the catheter tip is completed in sub-assembly form prior to installation in a catheter shaft sub-assembly, the connectors 292 may extend from the catheter tip sub-assembly to facilitate coupling to another flexible circuit, or lead wires extending from the catheter shaft sub-assembly. To further facilitate assembly, the connectors 292 may be electrically coupled to the flexible circuit(s) of the catheter shaft sub-assembly via an electrical connector. Alternatively, solder pads of the two flexible circuits may be soldered to one another. The use of flexible circuits may also further facilitate automation of the catheter assembly process.

In FIG. 2, electrical signals from distal and proximal thermocouples 68 and 68′ on flexible circuit 290 are isolated from one another by extending traces 296 on flexible circuit board 291 from the proximal thermocouples 68′ to solder pads 293_{1-N} on connector 292₁, and traces 296 from the distal thermocouples 68 to solder pads 291_{1-N} on connector 2922. This example circuit board routing mitigates electrical and electromagnetic cross-talk (interference) between the un-shielded electrical traces. The various electrical traces on the flexible circuit board 291 form a communication pathway.

In various embodiments, flexible circuit 290 may further include one or more electrical contacts 294₁₋₃ (for electrically coupling to spot electrodes). These electrodes, when capacitively coupled to a conductive shell, or extending through the conductive shell, may collect electrophysiology data related to tissue (e.g., myocardial tissue) in contact with (or in close proximity to) the conductive shell/electrodes. This electrophysiology data is then communicated via traces 296 to one or more solder pads 291 and 293 on the connectors 292 of the flexible circuit.

To facilitate coupling of flexible circuit 290 to a tip insert or other structure, vias 295 may extend through the flexible circuit board 291. In such embodiments, a protrusion may extend out from an external surface of a tip insert, and extend through the mating vias 295 in the flexible circuit board 291. Once properly located, the protrusions may be heat staked to create an interference fit between the via and the protrusion to permanently couple them. In the alternative, the flexible circuit board 291 may include bonding locations that facilitate such coupling. It is to be understood that various coupling means may be utilized, including: ultrasonic welding, fasteners, adhesives, friction and compression fits, etc. to achieve coupling of the flexible circuit board 291 to the tip insert. To facilitate electrical and thermal coupling between thermocouples 68 and 68′, and an inner surface of a conductive shell, the thermocouples may be directly coupled to the conductive shell. Thereby obviating any precise fitting required between the thermocouples and the conductive shell. In various embodiments consistent with the present disclosure, a quick thermal response of the thermocouples is desirable to provide an ablation control system with control inputs with as little lag as possible. Slow thermal response of the thermocouples may cause over ablation of tissue.

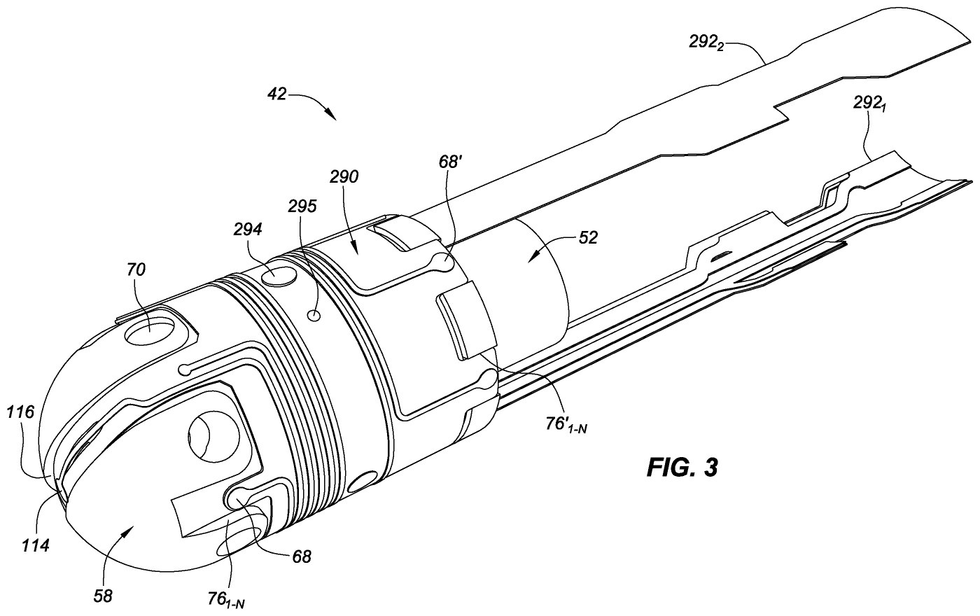

As discussed in more detail in relation to FIG. 3, when flexible circuit 290 is wrapped around a tip insert, distal thermocouples 68 form a first circumferentially-extending ring positioned near a tip of the catheter. Similarly, proximal thermocouples 68′, form a second circumferentially-extending ring positioned near a proximal end of the tip insert. Thermocouple 114 is wrapped around a distal radius of the tip insert, and is the distal most thermocouple.

It is to be understood that various circuit board layouts may be utilized to facilitate application specific design constraints in various flexible circuit 290 designs, consistent with the present disclosure. For example, to limit circuit board area, additional PCB layers may be added where the Z-dimension of a given application allows. Similarly, more or less connectors 292 may be implemented.

In various embodiments, the flexible circuit board 291 may include three layers: a copper layer at a top surface, an intermediate polyimide layer, and a constantan layer opposite the copper layer. Each of the thermocouples 68 and 68′ may be formed by drilling a via through the copper, polyimide, and constantan layers, and through plating the via with copper. Various thermocouple designs and manufacturing methods are well known in the art and may be applied hereto. Either side of the thermocouple is then electrically coupled to a trace on its respective layer. The voltage across the two traces may be compared, and the resulting voltage change is indicative of a temperature of a conductive shell thermally coupled to the thermocouple. In various applications, including ablation therapies, as the conductive shell is in direct contact with tissue being ablated, efficacy of an ablation therapy may be surmised.

In the present embodiment, flexible circuit 290 is designed to facilitate individual addressability of each of the thermocouples 68 and 68′, and electrical contacts 294. In more simplified embodiments, the thermocouples 68 in a distal circumferential ring may be electrically coupled in parallel to effectively facilitate temperature averaging of the distal thermocouples, and to minimize printed circuit board size. A similar configuration may also be utilized with thermocouples 68′. Such an embodiment may be particularly useful in applications where determining a tissue contact point along a circumference of the ablation catheter is not necessary. The present embodiment may also limit the effect of minute hot zones on an ablation control system.

As further shown in FIG. 2, each thermocouple (68 and 68′) is positioned on a small protrusion extending from a body of flexible circuit board 291. Each of the protrusions facilitate positive positioning of the flexible circuit board when assembled to a tip insert which has mating channel features (76 and 76′, as shown in FIG. 3), thereby preventing movement of the flexible circuit board relative to the tip insert. Such movement may otherwise affect thermal coupling of the thermocouples to an inner surface of a conductive shell. Similarly, thermocouple 114 also extends out onto a larger protrusion of the flexible circuit board, facilitating placement of the thermocouple 114 at the distal most tip of the catheter.

In some embodiments of flexible circuit 290, a top copper layer is placed above the two other layers of the flexible circuit board 291—polyimide, and constantan layers. Signal traces 296A, printed on the top copper layer, which are electrically coupled to a hot junction for each of the thermocouples. As is well known in the arts, thermocouples typically comprise two dissimilar metals joined together at respective ends of the dissimilar metals. The end of the thermocouple placed into thermal contact with a hot object is called the hot junction, while the opposite end, which is disposed to a base-line temperature within the tip insert, is a cold junction. The hot junction in the top copper layer and the cold junction in the constantan layer are electrically coupled to one another through the polyimide layer. When a catheter tip, consistent with the present embodiment, is placed against a warm object, such as myocardial tissue being ablated by radio frequencies, a voltage difference across the hot and cold junctions develops. The voltage difference is correlated with a temperature of the hot junction. The materials of the hot and cold junctions may include one or more of the following materials: iron, nickel, copper, chromium, aluminum, platinum, rhodium, alloys of any of the above, and other metals with high conductivity.

As each spot electrode (which is electrically coupled to one of electrical contacts 294₁₋₃) forms only one half of a circuit, each electrode need only one trace 296 extending to a connector 292 of the flexible circuit. The electrical signal from each spot electrode is compared and analyzed to detect electrophysiological characteristics indicative of medical conditions, such as, atrial fibrillation. Similarly, during and after treatment, the electrodes may be used to conduct diagnostics and determine a treatment efficacy.

In the embodiment of FIG. 2, all of the cold junctions may be electrically interconnected, and effectively function as a common ground for each of the thermocouples. By electrically interconnecting each of the electrical traces extending from the cold junctions, the number of common connector pads 293_{1-N} may be greatly reduced. As is envisioned in the present embodiment, the common ground for all of the thermocouples would require only a single connector pad, reducing circuit board 291 size and complexity.

FIG. 3 depicts an isometric front view of a partial catheter tip assembly 42 including a tip insert 58 with a flexible circuit 290 (as shown in FIG. 2) wrapped circumferentially around the ablation tip insert, consistent with various aspects of the present disclosure. Each of the distal thermocouples 68 may be located within a channel 76_{1-N}, and each of the proximal thermocouples 68′ may be located with a channel 76′_{1-N}. Each of the distal thermocouples 68 being positioned between lateral irrigation channels 70, which are circumferentially distributed about the tip insert 58. Distal tip channel 116 receives a distal tip thermocouple 114. To further facilitate coupling of the flexible circuit 290 to the tip insert 58, mating vias 295 through flexible circuit board 291 may receive protrusions that extend out from the insert. Once properly located, the protrusions may be heat staked to create an interference fit between the via and the protrusion to permanently couple them, or be otherwise coupled.

As further shown in FIG. 3, partial catheter tip assembly 42 includes one or more connectors 292₁₋₂ extending in a proximal direction from the main body portion of flexible circuit 290 and past shank 52. The connectors 292 may be long enough to be routed through an entire length of a catheter shaft, or may be a length that facilitates coupling the connectors 292 to another connector or a plurality of lead wires extending a length of the catheter shaft.

The tip insert 58 includes six laterally-extending irrigation channels 70, each of the irrigation channels 70 have a longitudinal axis arranged substantially perpendicular to the longitudinal axis of the catheter. The laterally-extending irrigation channels 70 deliver irrigant circumferentially about the catheter distal tip. It should be noted that the laterally-extending irrigation channels could be arranged at a different angle (i.e., different from 90°) relative to the catheter longitudinal axis. Also, more or fewer than six laterally-extending irrigation channels may be present in the tip insert.

In various embodiments, to assist longitudinal and radial placement of the flexible circuit relative to the tip insert, the tip insert may include longitudinally extending and radially offset channels 76_{1-N} and 76′_{1-N} on both proximal and distal ends (or just one end) of the tip insert.

The isometric orientation of the tip insert 58 in FIG. 3 reveals an arc-shaped channel 116 that extends toward the distal-most end to position a distal-most thermal sensor 114 at that location. It should be kept in mind that not all embodiments of the present disclosure will include this arc-shaped channel extension; however, a number of advantages may be realized by positioning a thermal sensor as far distally on the catheter tip as possible. For example, in view of the rapid heat dissipation experienced by the catheter tip, it can be desirable to sense temperature at this distal most location since it may be in the best location for most accurately determining the temperature of the surrounding tissue during certain procedures.

The tip insert 58 can be constructed from, for example, plastic (such as PEEK, which is polyether ether ketone) thermally-insulative ceramic (or other material with similar insulative properties), or ULTEM. All of the ablation tip inserts described herein are preferably constructed from thermally-insulative material.

Further, it should be understood that, in other embodiments of the thermally-insulative ablation tip insert (both irrigated and non-irrigated embodiments), there may be more or fewer channels 76. In fact, although the channels may facilitate placement of the sensors 68 on the insert (e.g., during catheter assembly), the outer surface of the main body of the tip insert may be smooth (or at least channel-less). In such an embodiment, the sensors may be aligned on the smooth outer surface of the tip insert (and, possibly, held in place by, for example, adhesive). Then, when the conductive shell is installed around the tip insert and the sensors 68, the gaps or voids between the inner surface of the conductive shell and the outer surface of the tip insert may be filled with material (e.g., potting material or adhesive). It is worth noting that the sensors may be put in place before or after the conductive shell is placed over the tip insert. For instance, the sensors may be mounted on (e.g., adhered to) the smooth outer surface of the tip insert forming a tip-insert-sensor subassembly. Then, the conductive shell may be placed over that tip-insert-sensor subassembly before the remaining voids between the tip-insert-sensor subassembly and the conductive shell are filled. Alternatively, the conductive shell may be held in place over the tip insert while one or more sensors are slid into the gap between the outer surface of the tip insert and the inner surface of the conductive shell. Subsequently, the voids would again be filled. These alternative manufacturing techniques may apply to all of the disclosed embodiments that comprise sensors mounted between a tip insert and a conductive shell member.

It should also be noted that the outer surface of the temperature sensors are mounted so as to at least be in close proximity to, and preferably to be in physical contact with, the inner surface of the conductive shell 44. As used herein, “in close proximity to” means, for example, within 0.0002 to 0.0010 inches, particularly if a conductive adhesive or other bonding technique is used to bond the temperature sensors to the inner surface of the shell. Depending on the specific properties of the sensors, the construction and materials used for the shell, and the type of conductive adhesive or the other bonding technique employed, it is possible that enough temperature sensitivity may be achieved despite even larger gaps between the sensors and the conductive shell, as long as the sensors are able to readily sense the temperature of the tissue that will be touching the outer surface of the conductive shell during use of the catheter tip.

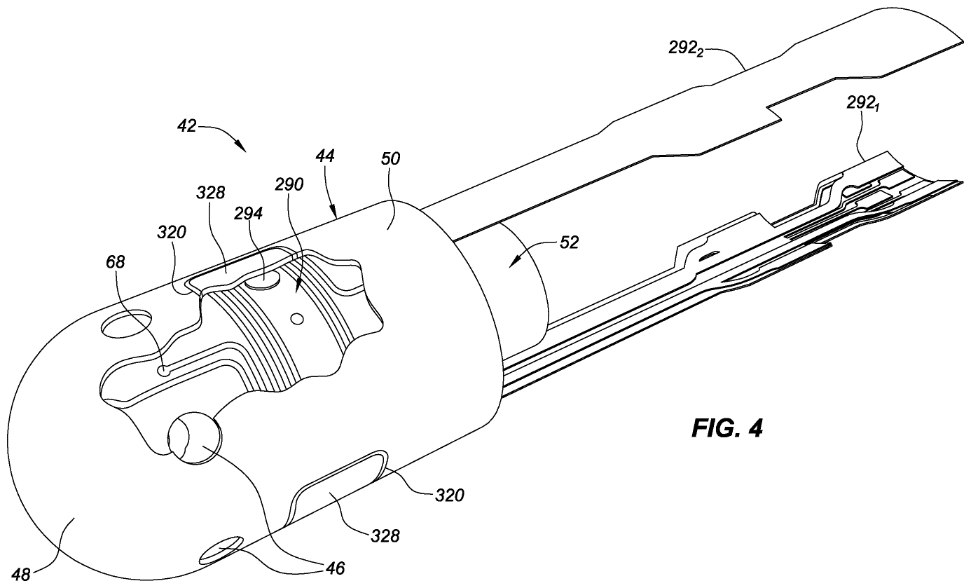

FIG. 4 depicts an isometric front view, including a partial cut-out, of the catheter tip assembly 42 of FIG. 3 with a conductive shell 44 encompassing at least a portion of a tip insert and a flexible circuit 290, consistent with various aspects of the present disclosure. When the conductive shell 44 is installed over the partial catheter tip assembly 42, irrigation holes 46 are aligned with lateral irrigation channels 70, and thermocouples 68 and 68′ on flexible circuit 290 are placed into thermal contact with an inner diameter of the conductive shell 44.

In FIG. 4, electrical contacts 294 on flexible circuit 290 are positioned below, and in electrically conductive contact with, spot electrodes 328 on a surface of conductive shell 44. These spot electrodes 328, depending on the application, may be located across an outer surface of the conductive shell 44, including domed distal end 48. When the spot electrodes 328 are placed into contact with tissue (e.g., myocardial tissue), the spot electrode receives electrical signal information indicative of the health of the tissue, the strength and directionality of electrical signals being transmitted through the tissue, among other information that is useful to the clinician to diagnose, treat, and determine patient outcome.

Where catheter tip assembly 42 is an RF ablation catheter, to reduce RF-related interference to the signals received by spot electrodes 328, it may be advantageous to electrically isolate the spot electrodes, from the rest of conductive shell 44 and an RF emitter within the catheter tip assembly. Accordingly, the FIG. 4 embodiment includes electrically insulative material 320 that at least partially circumscribes spot electrodes 328 to prevent/limit RF-related signal interference from being received by the spot electrodes.

The various conductive shells 44, disclosed herein, may comprise platinum, a platinum iridium composition, or gold. The conductive shell 44 (which may weigh, for example, 0.027 g) may comprise one or more parts or components. As shown in FIG. 4, the conductive shell may comprise a hemispherical or nearly-hemispherical domed distal end 48 and a cylindrical body 50. In one embodiment, the wall thickness of the conductive shell is 0.002 inches, but alternative wall thicknesses may also be utilized. The conductive shell may be formed or manufactured by, for example, forging, machining, drawing, spinning, or coining. Also, the conductive shell could be constructed from molded ceramic that has, for example, sputtered platinum on its external surface. In another alternative embodiment, the conductive shell could be constructed from conductive ceramic material.

Although a single-layer conductive shell 44 constructed from a thin layer of gold, for example, may perform in an magnetic resonance (MR) environment without causing undesirable or unmanageable MR artifacts, a conductive shell comprising an outer layer of a paramagnetic material such as platinum or platinum iridium, for example, may benefit from a multilayer construction as discussed below. A multilayer conductive shell may have just a multilayer cylindrical body portion, just a multilayer domed distal end portion, or both a multilayer domed distal end portion and a multilayer cylindrical body. Again, however, it is not a requirement that the domed distal end portion and the cylindrical body must both be constructed with the same number of layers or with the same thickness of layers. Also, the walls of the conductive shell 44 may, for example, be of a total thickness that is the same as, or nearly the same as, the thickness of the single-layer conductive shell 44 described above. The conductive shell may be formed or manufactured per, for example, the techniques already described herein.

Platinum iridium (a paramagnetic material) is commonly used for constructing catheter tips. Thus, various embodiments disclosed herein utilizing a thin conductive shell constructed entirely from platinum or platinum iridium (or some other paramagnetic material) may induce MR artifacts in an MR environment. Alternatively, for MR applications, the conductive tip shell may comprise a single layer constructed entirely from a diamagnetic material (e.g., a thin gold conductive shell) or a multilayer conductive shell including, for example, a platinum iridium outer layer and a diamagnetic material (e.g., gold or copper) inner layer. In such an embodiment, the paramagnetic outer layer and the diamagnetic inner layer minimize or entirely mitigate undesirable MR artifacts. Alternatively, the multilayer conductive shell may have an outer layer constructed from a diamagnetic material (such as bismuth or gold) and an inner layer constructed from a paramagnetic material (such as platinum or platinum iridium).

In yet another embodiment (not shown), a multilayer conductive shell may comprise more than two layers. For example, the conductive shell may comprise three layers, including a very thin outer layer of a paramagnetic material, a thicker intermediate layer of a diamagnetic material, and an oversized inner layer of a non-precious metal (or plastic or other material) sized to ensure that the finished geometry of the overall ablation tip is of a desired size for effective tissue ablation.

Materials that could be used for the inner layer include, but are not limited to, the following: silicon (metalloid); germanium (metalloid); bismuth (post transition metal); silver; and gold. Silver and gold are examples of elemental diamagnetic materials that have one-tenth the magnetic permeability of paramagnetic materials like platinum. Thus, one example multilayer shell configuration could comprise a platinum outer layer (or skin) and an inner layer (or liner or core) of gold or silver with a thickness ratio (e.g., platinum-to-gold thickness ratio) of at least 1/10 (i.e., the platinum layer being one-tenth as thick as the gold layer). In another example, a multilayer conductive shell configuration could comprise a platinum outer layer and a bismuth inner layer with a thickness ratio (e.g., platinum-to-bismuth thickness ratio) of at least ½ (i.e., the platinum outer layer being one-half as thick as the bismuth inner layer) since bismuth has a permeability that is about one-half the permeability of platinum. The layers may also be constructed from alloys, which may be used, for example, when a pure element material might otherwise be disqualified from use in the construction of a catheter tip.

In a typical ablation therapy for atrial fibrillation, pulmonary veins may be treated in accordance to their likelihood of having arrhythmic foci. Often, all pulmonary veins are treated. A distal tip of the catheter may include electrophysiology electrodes (also referred to as spot electrodes) which help to expedite diagnosis and treatment of a source of a cardiac arrhythmia, and may also be used to confirm a successful ablation therapy by determining the isolation of the arrhythmic foci from the left atrium, for example, or the destruction of the arrhythmic foci entirely.

During an ablation therapy, a distal end of an ablation catheter tip contacts ablation targeted myocardial tissue in order to conductively transfer energy (e.g., radio-frequency, thermal, etc.) thereto. It has been discovered that consistent force, during a series of tissue ablations, forms a more uniform and transmural lesion line. Such uniform lesion lines have been found to better isolate the electrical impulses produced by arrhythmic foci, thereby improving the overall efficacy of the ablation therapy. To achieve such consistent force, aspects of the present disclosure utilize a deformable body in the ablation catheter tip. The deformable body deforms in response to forces being exerted upon a distal end of the ablation catheter tip. The deformation of the deformable body may then be measured by a measurement device (e.g., ultrasonic, magnetic, optical, interferometry, etc.). Based on the tuning of the deformable body and/or the calibration of the measurement device, the deformation can then be associated with a force exerted on the distal end of the ablation catheter tip (e.g., via a lookup table, formula(s), calibration matrix, etc.).

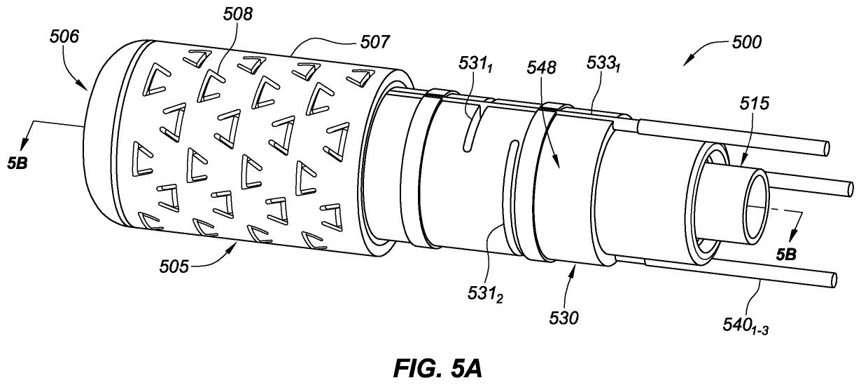

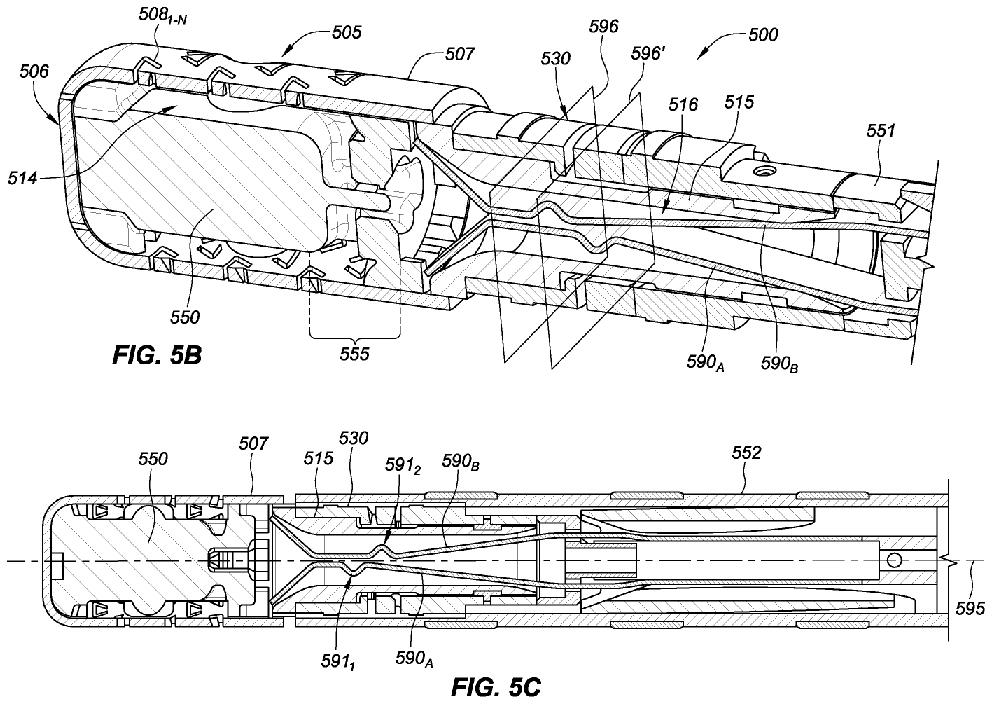

FIG. 5A is an isometric side view of a partial ablation catheter tip assembly 500, FIG. 5B is a isometric, cross-sectional side view of the partial ablation catheter tip assembly of FIG. 5A, and FIG. 5C is a cross-sectional side view of the partial ablation catheter tip assembly of FIG. 5A mounted to a distal end of a catheter shaft, consistent with various embodiments of the present disclosure.

Referring to FIGS. 5A-B, partial ablation catheter tip assembly 500 includes a flex tip 505 that is coupled to a distal end of a manifold 515. The manifold 515 may be comprised of, for example, a stainless steel alloy, MP35N (a cobalt chrome alloy), titanium alloy, or a composition thereof. The flex tip 505 includes a distal tip 506 and a flexible member 507. The flexible member 507 facilitates deformation of the flex tip in response to contact with tissue; more specifically, the flexible member 507 deforms to increase surface contact with target tissue. The increased tissue surface contact improves outcomes for various diagnostics and therapies (e.g., tissue ablation). After contact with target tissue is complete, the flexible member 507 returns to an un-deformed state. The distal tip 506 may be coupled to the flexible member 507 via an adhesive, weld, etc. A manifold 515, and an irrigation lumen 516 therein, extends through structural member 530, delivering irrigant from the irrigation lumen to a dispersion chamber 514 formed between the flex tip 505 and a tip insert 550.

In various embodiments of the present disclosure, to limit the deformation of a structural member 530, partial ablation catheter tip assembly 500 may transmit a portion of a force exerted on flex tip 505 through the manifold 515 (bypassing structural member 530). The manifold 515 transmits the force to a catheter shaft 552 that is coupled to a proximal end of the tip assembly 500 (as shown in FIG. 5C).

The cross-sectional side views of FIGS. 5B and 5C of the partial ablation catheter tip assembly 500 help to illustrate irrigant flow there through. The irrigant flows from an irrigant source through a catheter handle and into a central lumen of catheter shaft 552. The central lumen delivers the irrigant to a distal end of the catheter shaft. Upon arriving at the distal end of the catheter shaft the irrigant transitions into a smaller diameter irrigant lumen 516 of manifold 515 via end cap 551. Upon arriving at a proximal end of flexible member 507, a dispersion feature 555 distributes the irrigant circumferentially around tip insert 550 into a dispersion chamber 514 between the tip insert and the flexible member 507. The positive pressure within the dispersion chamber directs the irrigant radially out of the flex tip 505 via irrigant apertures 508_{1-N}.

In some embodiments, a flexible member 507 of flex tip 505 may comprise a titanium alloy (or other metal alloy with characteristics including a high tensile strength).

Structural member 530 houses a plurality of fiber optic cables 540₁₋₃ that extend through grooves, for example groove 533₁. In the present embodiment, the structural member 530 is divided into a plurality of segments along a longitudinal axis. The segments are bridged by flexure portions 531₁₋₂, each flexure portion defining a neutral axes. Each of the neutral axes constitute a location within the respective flexure portions where the stress is zero when subjected to a pure bending moment in any direction.

In a fiber optic distance measurement sensor, fiber optic cables 540₁₋₃ may be disposed in grooves 533, respectively, such that the distal ends of the fiber optic cables terminate at the gaps of either flexure portion 531₁₋₂. As shown in FIGS. 5A, and 5B, flexure portions 531₁₋₂ define a semi-circular segment that intercept an inner diameter of structural member 530. The flexure portions 531₁₋₂ may be formed by the various ways available to the artisan, such as but not limited to sawing, laser cutting or electro-discharge machining (EDM).

When a fiber optic sensor consistent with the above is assembled, one or more fiber optic cables 540 are mechanically coupled to structural member 530 via grooves 533. In some embodiments, each of the fiber optics may be communicatively coupled to a Fabry-Perot strain sensor within one of the gaps which form the flexure portions 531₁₋₂. The Fabry-Perot strain sensor includes transmitting and reflecting elements on either side of the slots to define an interferometric gap. The free end of the transmitting element may be faced with a semi-reflecting surface, and the free end of the reflecting element may be faced with a semi-reflecting surface.

In some assemblies of a fiber optic sensor, the fiber optic cables may be positioned along the grooves 533₁₋₃ (as shown in FIG. 5A) so that the respective Fabry-Perot strain sensor is bridged across one of the flexure portions 531₁₋₂. For example, a fiber optic cable may be positioned within a groove 533₁ so that the Fabry-Perot strain sensor bridges the gap at the flexure portion 5312.

In some embodiments, structural member 530 may comprise a composition including a stainless steel alloy (or other metal alloy with characteristics including a high tensile strength, e.g., titanium), or platinum iridium (e.g., in a 90/10 ratio).

In partial ablation catheter tip assembly 500 of FIGS. 5A and 5B, a trans-axial compliance of structural member 530 is corrected by directing a portion of a force exerted on flex tip 505 through manifold 515. By re-directing a portion of the trans-axial load onto the manifold 515, the resulting trans-axial deformation of the structural member 530 is reduced. In various embodiments of the present disclosure, the manifold may exhibit high deformation in response to axial force and reduced deformation in response to trans-axial forces. As a result, the manifold 515 will minimally increase the stiffness of the catheter tip assembly 500 in response to an axial force, while greatly increasing the stiffness in response to a trans-axial force. In some embodiments, the catheter tip assembly 500 may be tuned to target a 1:1 lateral-to-axial compliance ratio; for example, a 500:1 lateral-to-axial compliance ratio or less (1500 nanometers lateral motion to 3 nanometers axial).

Further referring to FIGS. 5A-C, the structural member 530 may be coupled at a distal end to a distal end of manifold 515, and at a proximal end to both the manifold 515 and an end cap 551. In some embodiments, the end cap may be made of platinum, titanium alloy, stainless steel alloy, MP35N (a cobalt chrome alloy), or a combination thereof. Once the tip assembly 500 is complete, the structural member 530 may be further coupled at a proximal end to a catheter shaft 552 (as shown in FIG. 5C) that extends proximally to a catheter handle. The structural member 530 is designed in such a way as to receive forces exerted on the flex tip 505 of the catheter tip assembly 500 and to absorb such force by deflecting and deforming in response thereto. Further, and as discussed in more detail above, the structural member 530 may be outfitted with a measurement device which facilitates measurement of the deflection/deformation of the deformable body which may be correlated with the force exerted on the flex tip 505 and communicated with a clinician. Knowledge of a force exerted on the flex tip 505 of a catheter may be useful for a number of different cardiovascular operations; for example, during a myocardial tissue ablation therapy it is desirable to know a contact force exerted by the flex tip 505 of the catheter on target tissue as the time to necrose tissue is based on energy transferred between the catheter and tissue—which is highly dependent upon the extent of tissue contact.

In the various catheter tip assemblies disclosed herein, various electronic components in the catheter tip are necessary to facilitate desired functionality. As discussed in more detail above, the catheter tip may include, for example, one or more radio-frequency ablation electrodes, one or more electrophysiology electrodes, and/or a plurality of thermocouples. All of these electronic components must be communicatively coupled to a computer system at a proximal end of the catheter (as discussed above in reference to FIG. 1). Prior art ablation catheter systems utilized individual lead wires, extending the length of the catheter shaft, to facilitate communication between the various distal tip components and the computer system. Aspects of the present disclosure are directed to reduced catheter assembly complexity by using one or more flexible circuits which extend at least a portion of the length of the catheter shaft, and communicatively couple the electronic components to the computer system.

In the embodiment disclosed in FIGS. 5A-C, flexible circuits 590A-B are routed through an irrigant lumen 516 of manifold 515, the same cross-sectional path taken by irrigant delivered to the dispersion chamber 514. The irrigant fluid flow path traverses through end cap 551 into the start of the shared space with the flexible circuits, within the irrigant lumen 516, the irrigant then flows around the flexible circuits in the irrigant lumen and within the dispersion chamber 514 before exiting through the irrigant apertures 508_{1-N}.

Importantly, it has been discovered that the flexible circuits 590₁₋₂ within the ablation catheter tip assembly 500 may function as a structural element of ablation catheter tip assembly 500 in some situations; for example, in response to axial deflections/deformations of the catheter tip. Moreover, in some applications one or more of the flexible circuits, depending on their relative placement to a longitudinal axis of the catheter shaft may also function as a structural element in response to lateral deflections of the catheter tip. This is particularly problematic in ablation catheter systems capable of force sensing (such as discussed herein), and may affect the accuracy of the force measurement system. To address such problems, the present embodiment utilizes a formed bend 591₁₋₂ in one or more of the flexible circuits, which is positioned within the irrigant lumen 516 of manifold 515. As a result, the formed bends in the flexible circuits readily deflect in response to an axial deflection on the catheter tip, absorbing very little of the force, and allowing the force to be almost completely transmitted to the structural member 530, which will deform, the deformation will be measured, and the force exerted on the catheter tip extrapolated therefrom.

Moreover, as each of the flexible circuits have a primarily rectangular cross-section, the flexible circuits are more rigid along a horizontal plane (also referred to as a non-flexible plane, less-flexible plane, less pliable plane), in response to a torque; whereas the flexible circuits are more pliable along vertical planes (also referred to as a flexible plane or more pliable plane). The flexible circuits, along the vertical planes 596 and 596′, are made more pliable due to the formed bends 591₁₋₂. The flexible circuits are far more rigid along horizontal plane 595. Similarly, in many embodiments, structural member 530 may also exhibit varying degrees of flexibility depending on the force vector applied to the conductive shell. The variable flexibility of the structural member may be due, at least in part, to the structural member lacking symmetry across one or more planes extending through a longitudinal axis of the structural member. As a result, a radial vector (or composite radial vector) of the force exerted on the flex tip may greatly impact the resulting deformation of structural member 530. To (at least partially) correct for the resulting lack of repeatability in the deflection of the structural member in response to a constant lateral force exerted with varying radial vectors (and the resulting force measurement calculations), a less pliable plane of the structural member may be aligned with one of the pliable vertical planes 596 and 596′, and if possible a more pliable plane of the structural member may be aligned with a less pliable plane of the flexible circuits (e.g., horizontal plane 595).

In various embodiments of the catheter tip assemblies disclosed herein, the catheter tip assemblies may also include a plurality of spot electrodes on a conductive shell thereof which facilitate electrophysiology mapping of tissue, such as myocardial tissue, in (near) contact with the shell. In more specific embodiments, the plurality of spot electrodes may be placed across the shell in such a manner as to facilitate Orientation Independent Algorithms which enhance electrophysiology mapping of the target tissue and is further disclosed in U.S. application Ser. No. 15/152,496, filed 11 May 2016, U.S. application Ser. No. 14/782,134, filed 7 May 2014, U.S. application Ser. No. 15/118,524, filed 25 Feb. 2015, U.S. application Ser. No. 15/118,522, filed 25 Feb. 2015, and U.S. application No. 62/485,875, filed 14 Apr. 2017, all of which are now pending, and are incorporated by reference as though fully disclosed herein.

While various embodiments of the present disclosure, including FIGS. 2-4, are directed to ablation catheter tips including two rings of 6 radially-disposed thermal sensors and one distal thermal sensor placed close to the distal end of the catheter tip, the invention is not limited to such a thirteen-sensor configurations. Various other configurations are readily envisioned.

It is to be understood that while an irrigated ablation catheter tip is illustrated in various embodiments of the present disclosure, the design of the structural assembly (including structural member, manifold, and end cap) is modular and may facilitate the fitting of various catheter tips (e.g., rigid, flex, and other advanced irrigation tips).

Applicant further envisions utilizing catheters comprising various segmented tip designs with the ablation catheter system described above. Example tip configurations are disclosed in U.S. patent application No. 61/896,304, filed 28 Oct. 2013, and in related international patent application no. PCT/US2014/062562, filed 28 Oct. 2014 and published 7 May 2015 in English as international publication no. WO 2015/065966 A2, both of which are hereby incorporated by reference as though fully set forth herein.

Although several embodiments have been described above with a certain degree of particularity, those skilled in the art could make numerous alterations to the disclosed embodiments without departing from the present disclosure. It is intended that all matter contained in the above description or shown in the accompanying drawings shall be interpreted as illustrative only and not limiting. Changes in detail or structure may be made without departing from the present teachings. The foregoing description and following claims are intended to cover all such modifications and variations.

Various embodiments are described herein of various apparatuses, systems, and methods. Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

Reference throughout the specification to “various embodiments,” “some embodiments,” “one embodiment,” “an embodiment,” or the like, means that a particular feature, structure, or characteristic described in connection with the embodiment is included in at least one embodiment. Thus, appearances of the phrases “in various embodiments,” “in some embodiments,” “in one embodiment,” “in an embodiment,” or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation.

It will be appreciated that the terms “proximal” and “distal” may be used throughout the specification with reference to a clinician manipulating one end of an instrument used to treat a patient. The term “proximal” refers to the portion of the instrument closest to the clinician and the term “distal” refers to the portion located furthest from the clinician. It will be further appreciated that for conciseness and clarity, spatial terms such as “vertical,” “horizontal,” “up,” and “down” may be used herein with respect to the illustrated embodiments. However, surgical instruments may be used in many orientations and positions, and these terms are not intended to be limiting and absolute.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A high-thermal-sensitivity ablation catheter tip, the tip comprising:
a conductive shell including a dispersion chamber configured and arranged for irrigant distribution;
a structural member coupled to a proximal end of the conductive shell, the structural member configured and arranged to deflect in response to a force exerted on the conductive shell;
a manifold including an irrigation lumen extending through a longitudinal axis of the manifold, the irrigation lumen configured and arranged to deliver irrigant into the dispersion chamber; and
a flexible electronic circuit that extends through the irrigant lumen of the manifold.

2. The high-thermal-sensitivity ablation catheter tip of claim 1, wherein the flexible electronic circuit includes one or more bends positioned on a portion of the flexible circuit within the irrigant lumen, the one or more bends configured and arranged to deflect in response to an axial force exerted on the conductive shell while minimally absorbing the axial force.

3. The high-thermal-sensitivity ablation catheter tip of claim 1, wherein the flexible electronic circuit is configured and arranged to communicatively couple a computer system at a proximal end of an ablation catheter and one or more electronic components within the conductive shell.

4. The high-thermal-sensitivity ablation catheter tip of claim 3, wherein the one or more electronic components include at least one of the following: thermal sensors, electrophysiology electrodes, and radio-frequency electrodes.

5. The high-thermal-sensitivity ablation catheter tip of claim 1, wherein the structural member is configured and arranged with non-uniform flexibility, and the flexible electronic circuit is configured and arranged with non-uniform flexibility, and wherein a flexible plane of the flexible electronic circuit and a less-flexible plane of the structural member are aligned about the longitudinal axis.

6. The high-thermal-sensitivity ablation catheter tip of claim 5, wherein the flexible plane of the flexible electronic circuit is associated with a bend on the flexible electronic circuit, the bend configured and arranged to counteract the effect of the less-flexible plane of the structural member.

7. The high-thermal-sensitivity ablation catheter tip of claim 1, wherein the structural member is further configured and arranged to absorb a first portion of a lateral force exerted on the conductive shell, and the manifold is configured and arranged to absorb a second portion of the lateral force exerted on the conductive shell, and wherein the manifold and the structural element, as combined, have a lateral-to-axial compliance ratio less than 500:1.

8. The high-thermal-sensitivity ablation catheter tip of claim 1, further including
a thermally-insulative tip insert, wherein the conductive shell surrounds at least a portion of the tip insert; and
wherein the flexible electronic circuit is wrapped around the tip insert, and includes a plurality of thermal sensors in thermal communication with the conductive shell,
and distributed across at least one of a length and width of the flexible electronic circuit, and
a communication pathway at least partially disposed on the flexible electronic circuit, communicatively coupling the plurality of thermal sensors to a computer system.

9. The high-thermal-sensitivity ablation catheter tip of claim 8, wherein the flexible electronic circuit further includes a plurality of electrophysiology electrodes positioned in electrical isolation from the conductive shell, and communicatively coupled to the computer system via the communication pathway.

10. The high-thermal-sensitivity ablation catheter tip of claim 8, wherein the plurality of thermal sensors are configured in two circumferential rings around the tip insert, where a first circumferential ring is longitudinally offset relative to a second circumferential ring.

11. The high-thermal-sensitivity ablation catheter tip of claim 10, wherein the plurality of thermal sensors further includes an additional thermal sensor positioned near a distal-most end of the tip insert.

12. The high-thermal-sensitivity ablation catheter tip of claim 9, wherein the electrophysiology electrodes are spot electrodes, and the spot electrodes extend through apertures in the conductive shell, each of the spot electrodes circumscribed by an electrically insulative material configured to reduce signal interference between the conductive shell and the spot electrode.

13. The high-thermal-sensitivity ablation catheter tip of claim 8, wherein the tip insert comprises a material selected from the group consisting of plastic, ceramic, and a material with similar insulative properties to a ceramic.

14. The high-thermal-sensitivity ablation catheter tip of claim 1, wherein the structural member comprises a material selected from the group consisting of a stainless steel alloy, titanium alloy, and platinum iridium.

15. The high-thermal-sensitivity ablation catheter tip of claim 1, wherein the manifold comprises a material selected from the group consisting of a stainless steel alloy, a cobalt chrome alloy, and a titanium alloy.

16. The high-thermal-sensitivity ablation catheter tip of claim 1, wherein the conductive shell comprises a material selected from the group consisting of platinum, a platinum iridium composition, and gold.

17. A method of assembling an ablation catheter tip, the method comprising:
providing a manifold with an irrigant lumen extending there through;
providing a flexible electronic circuit including one or more thermocouples; and
directing a distal portion of the flexible circuit through the irrigant lumen.

18. The method of claim 17, further including
forming a bend in the flexible electronic circuit, and
positioning the bend within the irrigant lumen of the manifold.

19. The method of claim 17, wherein the flexible electronic circuit includes at least one flexible plane; and the method further including
providing a structural member with at least one less-flexible plane,
circumferentially encompassing at least a portion of the manifold with the structural member, and
radially aligning the at least one flexible plane of the flexible electronic circuit and the less-flexible plane of the structural member about a longitudinal axis of the ablation catheter tip.

20. The method of claim 17, further including providing a tip insert;
wrapping the distal portion of the flexible circuit about the tip insert;
providing a conductive shell; and
inserting the tip insert and the distal portion of the flexible circuit into the conductive shell, thereby placing the one or more thermocouples into thermally-transmissive contact with an inner surface of the conductive shell.
